(19) United States
(12) Patent Application Publication
(10) Pub. No.: US 2024/0279177 A1
(43) Pub. Date: Aug. 22, 2024

(54) PREPARATION OF A CLASS OF LYSINE-SPECIFIC DEMETHYLASE 1 (LSD1) INHIBITORS

(71) Applicants: JIANGSU LIANHUAN PHARMACEUTICAL CO., LTD., Yangzhou (CN); SHANGHAI INSTITUTE OF MATERIA MEDICA, CHINESE ACADEMY OF SCIENCES, Shanghai (CN)

(72) Inventors: Hong LIU, Yangzhou (CN); Ben NIU, Shanghai (CN); Chunpu LI, Yangzhou (CN); Jianping CHEN, Yangzhou (CN); Kun HUANG, Shanghai (CN); Jiang WANG, Yangzhou (CN)

(21) Appl. No.: 18/392,472

(22) Filed: Dec. 21, 2023

(30) Foreign Application Priority Data
Dec. 22, 2022 (CN) 202211657811.X

(51) Int. Cl. C07D 211/60; C07D 401/12; C07D 409/12

(52) U.S. Cl. CPC C07D 211/60; C07D 401/12; C07D 409/12

(57) ABSTRACT

The present invention relates to the fields of medicinal chemistry and pharmaco therapeutics, and specifically to a method for preparing a compound of formula I and a chemically acceptable salt thereof.

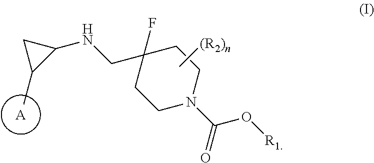

### TECHNICAL FIELD

The present invention belongs to the field of pharmaceutical synthesis technology, and specifically relates to the method for preparing a class of lysine-specific demethylase 1 (LSD1) inhibitors.

### BACKGROUND

Post-translational modification of histones is an important area of epigenetic research, and aberrant histone modifications are closely related to tumour development. LSD1 (also known as BHC_{110,} p110b and NPAO), identified by Shi's group in 2004, is the first identified histone demethylase and its structure is highly conserved from yeast to human. In normal haemocytes, LSD1 inhibits haematopoietic cell differentiation by transcriptionally repressing the expression of downstream target genes through binding to haematopoietic-associated transcription factors (e.g. GFI1B or TAL1). LSD1 plays a key role in dynamically regulating the degree of differentiation of normal haematopoietic cells; however, when this dynamic regulation is imbalanced, the process of haematopoietic cell differentiation is disrupted, which can lead to the development of leukaemia. Studies have shown that LSD1 is highly expressed in the bone marrow of 90% of acute myeloid leukemia (AML) and 78% of acute T-cell acute lymphoblastic leukemia (T-ALL) cases. The expression of LSDI was significantly higher in poorly differentiated AML cells than in more differentiated AML cells. In a mouse model of MLL-AF9 fusion leukaemia, LSD1 was found to be essential for maintaining the stemness of leukaemia stem cells, inhibiting haematopoietic cell differentiation, and promoting excessive cell proliferation. When LSD1 gene was knocked down, AML stem cells lost stemness by inducing differentiation and apoptosis, and were unable to achieve clone formation in vitro and tumour transplantation process in vivo. Meanwhile, LSD1 is aberrantly expressed in a variety of solid tumour cells and tissues, which is closely associated with poor prognostic responses. Therefore, LSD1 has emerged as a potent and epigenetically related anti-tumour target, and the development and synthesis of structurally novel small molecule LSD1 inhibitors have great research importance for the treatment of diseases such as malignant tumours and leukaemia.

### SUMMARY

The purpose of the present invention is to provide a method for preparing LSD1 inhibitors with easier implementation and higher yield.

In a first aspect of the present invention, provided is a method for preparing a compound of Formula I, or a pharmaceutically acceptable salt thereof:

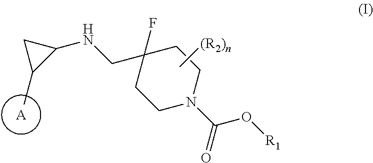

wherein,

ring A is selected from the group consisting of: substituted or unsubstituted C₆-C₁₀ aromatic ring, and substituted or unsubstituted 5-12 membered heteroaromatic ring;

R₁ is selected from the group consisting of: —CH₂—R; wherein R is selected from the group consisting of: H, C₁-C₄ alkyl, substituted or unsubstituted C₆-C₁₀ aryl, substituted or unsubstituted C₃-C₈cycloalkyl, substituted or unsubstituted 5- to 10-membered heteroaryl, substituted or unsubstituted 4- to 10-membered heterocyclyl; wherein the “substituted” refers to one or more hydrogen atoms on the group being substituted by groups selected from the group consisting of: halogen, C₁-C₄ alkyl, hydroxyl, carboxyl, C₁-C₄alkoxy, C₁-C₄haloalkyl, and amino-protecting group; wherein the heteroaryl or heterocyclyl comprises 1, 2, or 3 heteroatoms selected from the group consisting of: N, O, and S; and wherein the amino-protecting group is selected from the group consisting of: Boc, SEM, Cbz, Fmoc, Alloc, Teoc, Tos, Tfa, PMB and Bn;

each R₂ is independently selected from the group consisting of: methyl;

n is 0, 1 or 2;

the method comprises the steps:

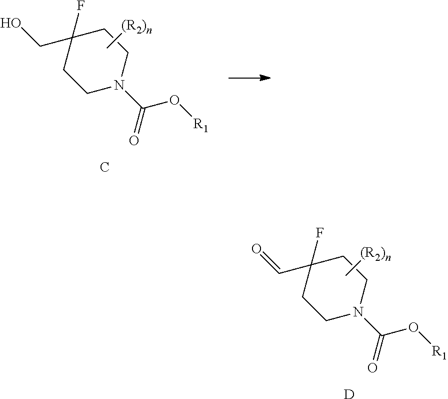

(1) In a solvent, converting compound C into compound D in the presence of an oxidizing agent;

wherein the oxidising agent is selected from the group consisting of: potassium chromate sulfate, pyridine chromium trioxide/pyridine, pyridine chromium trioxide, pyridine chlorochromic acid, oxalyl chloride/dimethylsulfoxide, pyridine sulfur trioxide/dimethylsulfoxide/triethylamine, Dess-Martin high iodine oxidiser, and iodosobenzene diacetate/TEMPO;

the solvent is selected from the group consisting of: C₁-C₄ halogenated hydrocarbon solvent, acetone, acetonitrile, tetrahydrofuran, ethyl acetate, toluene, dioxane, and combinations thereof;

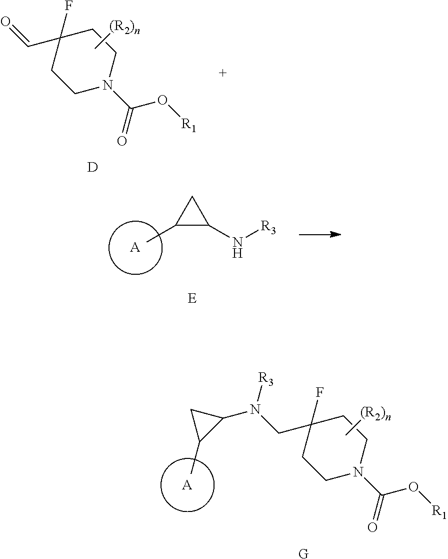

(2) Compound D and Compound E undergo reductive amination to produce Compound G.

In another preferred embodiment, the step (2) is carried out in the presence of a reducing agent and an additive.

In another preferred embodiment, the reducing agent is selected from the group consisting of: sodium cyanoborohydride, sodium triacetoxyborohydride, sodium borohydride, and combinations thereof.

In another preferred embodiment, the additive is selected from the group consisting of: formic acid, acetic acid, and combinations thereof.

In another preferred embodiment, the step (2) is carried out in a solvent selected from the group consisting of: C₁-C₄ halogenated hydrocarbon solvent, C₁-C₆ alcoholic solvent, tetrahydrofuran, 2-methyltetrahydrofuran, ethyl acetate, isopropyl acetate, and combinations thereof.

In another preferred embodiment, R is selected from the group consisting of: substituted or unsubstituted phenyl, substituted or unsubstituted C₅-C₆cycloalkyl, substituted or unsubstituted 5-7-membered heteroaryl, and substituted or unsubstituted 5-6-membered heterocyclyl; wherein the “substituted” refers to one or more hydrogen atoms on the group being substituted by groups selected from the group consisting of: halogen, C₁-C₄ alkyl, hydroxyl, carboxyl, C₁-C₄alkoxy, C₁-C₄ haloalkyl, and amino-protecting group; the heteroaryl or heterocyclyl comprises 1, 2, or 3 heteroatoms selected from the group consisting of: N, O, and S; and the amino-protecting group is selected from the group consisting of: Boc, SEM, Cbz, Fmoc, Alloc, Teoc, Tos, Tfa, PMB, and Bn.

In another preferred embodiment, R₁ is selected from the group consisting of: ethyl, benzyl, phenyl, cyclohexylmethyl, 4-pyridinylmethyl, phenylethyl, 1H-indole-5-ylmethyl, 2-thienylmethyl, 2-furyl methyl, 4-fluorophenylmethyl, 4-chlorophenylmethyl, 4-bromophenylmethyl, 4-methoxyphenyl, 4-trifluoromethylphenylmethyl, 3,5-dimethoxyphenylmethyl, 4-carboxyphenylmethyl, cyclopentyl methyl, cyclobutylmethyl, 4-piperidyl methyl, 3-chlorophenylmethyl, 2-chlorophenylmethyl, 4-tert butylphenylmethyl.

In another preferred embodiment, the compound of formula G is selected from the group consisting of:

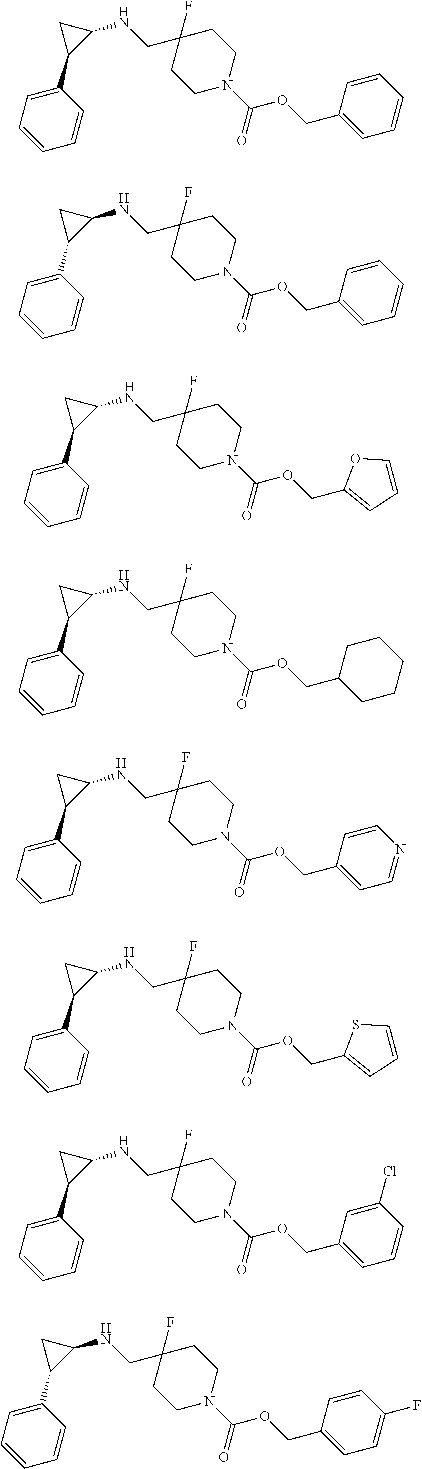

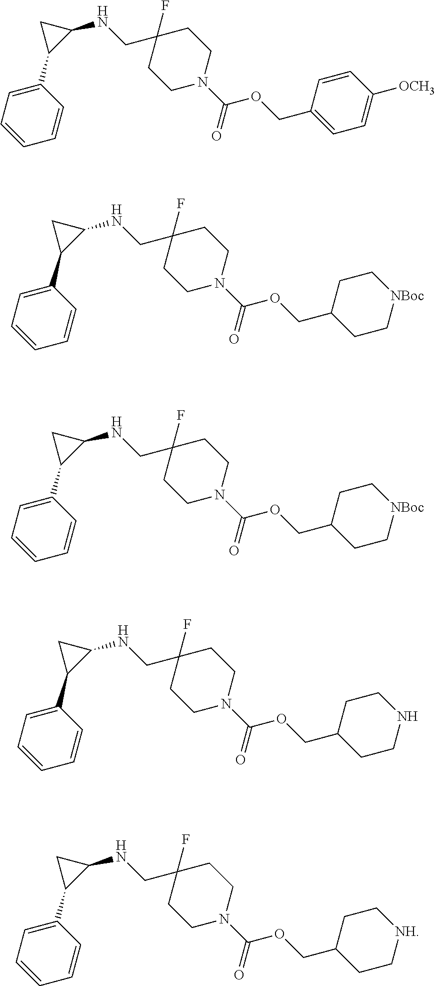 [SMILES: COc1ccc(COC(=O)N2CCC(F)(CN[C@@H]3C[C@H]3c3ccccc3)CC2)cc1 ; CC(C)(C)OC(=O)N1CCC(COC(=O)N2CCC(F)(CN[C@H]3C[C@@H]3c3ccccc3)CC2)CC1 ; CC(C)(C)OC(=O)N1CCC(COC(=O)N2CCC(F)(CN[C@@H]3C[C@H]3c3ccccc3)CC2)CC1 ; O=C(OCC1CCNCC1)N1CCC(F)(CN[C@H]2C[C@@H]2c2ccccc2)CC1 ; O=C(OCC1CCNCC1)N1CCC(F)(CN[C@@H]2C[C@H]2c2ccccc2)CC1]

In some embodiments, in step (1), the oxidising agent is selected from the group consisting of: oxalyl chloride/dimethylsulfoxide, pyridine sulfur trioxide/dimethylsulfoxide/triethylamine, Dess-Martin oxidiser, and iodosobenzene diacetate/TEMPO.

In some embodiments, in step (1), the solvent is a halogenated hydrocarbon solvent.

In some embodiments, in step (1), the solvent is dichloromethane.

In some embodiments, in step (1), the molar ratio of the compound of formula C to the oxidising agent is 1:1.0-1:6.0.

In another preferred embodiment, the temperature of the reaction is −80˜40° C.

In another preferred embodiment, the time of the reaction is 4-48 hours.

In another preferred embodiment, the ratio V/W of the volume of the reaction solvent used to the weight of the substrate is 5˜20 mL/g.

In some embodiments, the molar ratio of the compound of formula C to the oxidising agent is 1:2.0˜1:4.0.

In another preferred embodiment, the temperature of the reaction is −78 to 20° C.

In another preferred embodiment, the time of the reaction is 2-24 hours.

In some embodiments, in step (2), the reducing agent is selected from the group consisting of: sodium cyanoborohydride, and sodium triacetoxyborohydride.

In some embodiments, in step (2), the solvent is selected from the group consisting of: C₁-C₆ alcoholic solvent, and C₁-C₄ halogenated hydrocarbon solvent.

In some embodiments, in step (2), the molar ratio of the compound of formula D to the reducing agent is 1:0.8˜1:2.0; and/or

the molar ratio of the compound of formula D to the compound of formula E is 1:0.5˜1:2.0; and/or

the molar ratio of the compound of formula D to the additive is 1:0.5˜1:2.0.

In another preferred embodiment, the temperature of the reaction is −10 to 50° C.

In another preferred embodiment, the time of the reaction is 1-48 hours.

In another preferred embodiment, the ratio V/W of the volume of the reaction solvent used to the weight of the substrate is 5 mL/g˜30 mL/g.

In another preferred embodiment, the method includes the steps:

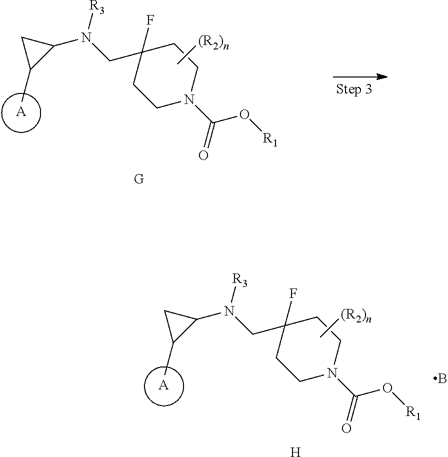

(3) Reacting compound G with an organic or inorganic acid to obtain compound H.

In another preferred embodiment, in step (3), the organic or inorganic acid is selected from the group consisting of: hydrochloric acid, hydrobromic acid, sulfuric acid, nitric acid, sulfamic acid, phosphoric acid, citric acid, tartaric acid, lactic acid, pyruvic acid, acetic acid, benzenesulfonic acid, p-toluenesulfonic acid, methanesulfonic acid, naphthalene sulfonic acid, ethanesulfonic acid, naphthalene disulfonic acid, maleic acid, malic acid, malonic acid, fumaric acid, succinic acid, propionic acid, oxalic acid, trifluoroacetic acid, stearic acid, pamoic acid, hydroxymaleic acid, phenylacetic acid, benzoic acid, salicylic acid, glutamic acid, ascorbic acid, p-aminobenzenesulfonic acid, 2-acetyloxybenzoic acid, hydroxyethanesulfonic acid, and combinations thereof.

In another preferred embodiment, in step (3), the solvent is selected from the group consisting of: methanol, ethanol, dimethyl sulfoxide, methyl tert-butyl ether, dioxane, acetone, acetonitrile, tetrahydrofuran, ethyl acetate, isopropyl acetate, and combinations thereof.

In another preferred embodiment, in step (3), the solvent is selected from the group consisting of: mixed solvent of C₁-C₆ alcoholic solvent/water, mixed solvent of acetonitrile/water, and mixed solvent of acetone/water.

In some embodiments, in step (3), the molar ratio of the compound of formula G to the salt-forming reagent is 1:1.0˜5.0.

In another preferred embodiment, in step (3), the temperature of the reaction is preferably 20-100° C.

In another preferred embodiment, in step (3), the time of the reaction is preferably 2-48 hours.

In another preferred embodiment, in step (3), the ratio V/W of the volume of the reaction solvent used to the weight of the substrate is 5 mL/g to 30 mL/g.

In a second aspect of the present invention, provided is a method for preparing intermediate compound of formula D:

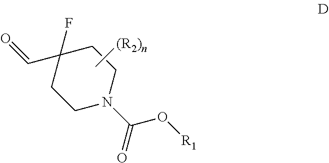

wherein,

R₁ is selected from the group consisting of: ethyl, benzyl, phenyl, cyclohexylmethyl, 4-pyridinylmethyl, phenylethyl, 1H-indole-5-ylmethyl, 2-thienylmethyl, 2-furyl methyl, 4-fluorophenylmethyl, 4-chlorophenylmethyl, 4-bromophenylmethyl, 4-methoxyphenyl, 4-trifluoromethylphenylmethyl, 3,5-dimethoxyphenylmethyl, 4-carboxyphenylmethyl, cyclopentyl methyl, cyclobutylmethyl, 4-piperidyl methyl, 3-chlorophenylmethyl, 2-chlorophenylmethyl, and 4-tert butylphenylmethyl;

each R₂ is independently selected from the group consisting of: methyl;

n is 0, 1 or 2;

the method comprises the steps:

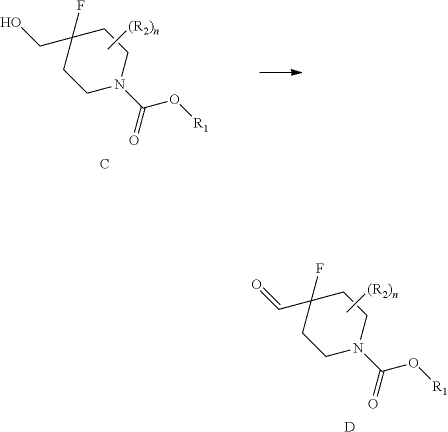

(1) In a solvent, converting compound C into compound D in the presence of an oxidizing agent;

wherein the oxidising agent is selected from the group consisting of: potassium chromate sulfate, pyridine chromium trioxide/pyridine, pyridine chromium trioxide, pyridine chlorochromic acid, oxalyl chloride/dimethylsulfoxide, pyridine sulfur trioxide/dimethylsulfoxide/triethylamine, Dess-Martin high iodine oxidiser, and iodosobenzene diacetate/TEMPO;

the solvent is selected from the group consisting of: C₁-C₄ halogenated hydrocarbon solvent, acetone, acetonitrile, tetrahydrofuran, ethyl acetate, toluene, dioxane, and combinations thereof.

It should be understood that within the scope of the present invention, the above-mentioned technical features of the present invention and the technical features specifically described in the following (such as embodiments) can be combined with each other to form a new or preferred technical solution. Limited by space, it will not be repeated here.

### DETAILED DESCRIPTION OF THE INVENTION

After long-term and in-depth research, the present inventors obtained a new method for synthesizing synthetizing tranylcypromine analogues. The synthetic method has the advantages of high yield, pure product and suitable for industrial production, and thus very suitable for producing the tranylcypromine analogues. The present invention has been completed on this basis.

### Definition

As used herein, the term “alkyl” includes a linear or branched alkyl. For example, C₁-C₆ alkyl represents a straight or branched alkyl with 1-6 carbon atoms, such as methyl, ethyl, propyl, isopropyl, butyl, isobutyl, tert-butyl, etc.

As used herein, the term “alkenyl” includes a straight or branched alkenyl. For example, C₂-C₆ alkenyl refers to a linear or branched alkenyl with 2-6 carbon atoms, such as vinyl, allyl, 1-propenyl, isopropenyl, 1-butenyl, 2-butenyl, or similar groups.

As used herein, the term “alkynyl” includes a straight or branched alkynyl.For example, C₂-C₆alkynyl refers to a straight or branched alkynyl with 2-6 carbon atoms, such as ethynyl, propynyl, butynyl, or similar groups.

As used herein, the term “cycloalkyl” refers to a cyclic saturated aliphatic hydrocarbon group having a specific number of carbon atoms. For example, C₃-C₁₀ alkenyl refers to a cyclic saturated aliphatic hydrocarbon group having 3-10 carbon atoms. It can be a single ring, such as cyclopropyl, cyclobutyl, cyclopentyl, cyclohexyl, or similar groups. It can also be in the form of a bicyclic ring, such as a bridged ring or a spiro ring.

As used herein, the term “alkylamino” refers to an amino substituted with alkyl group. For example, “C₁-C₈alkylamino” refers to an amino substituted with C₁-C₆ alkyl, which may be mono- or di- substituted; e.g., methyl-amino, ethyl-amino, propyl-amino, isopropyl-amino, butyl-amino, isobutyl-amino, tert-butyl-amino, dimethyl-amino, diethyl-amino, dipropyl-amino, diisopropyl-amino, dibutyl-amino, diisobutyl-amino, di-tert-butyl-amino, etc.

As used herein, the term “alkoxy” refers to a group having an alkyl-oxy structure. For example, “C₁-C₈alkoxy” refers to a straight or branched alkoxy having 1-8 carbon atoms, including methoxy, ethoxy, propoxy, isopropoxy, butoxy, isobutoxy, tert-butoxy, etc.

As used herein, the term “halogenated alkyl” represents an alkyl in which one or more hydrogen atoms have been substituted with halogens, wherein the alkyl is defined as above.

As used herein, the term “haloalkoxy” represents an alkoxy in which one or more hydrogen atoms have been substituted with halogens, wherein alkoxy is defined above.

As used herein, the term “heterocyclyl” or “heterocycloalkyl” refers to a saturated or partially saturated cyclic group having a specific number of ring atoms (e.g., 3-10 ring atoms) of which 1-3 atoms are heteroatoms selected from N, S, and O. It may be monocyclic, or bicyclic or polycyclic, e.g., in the form of a bridged ring or a spiro ring. Specific examples may be oxetanyl, azetidinyl, tetrahydro-2H-pyranyl, piperidinyl, tetrahydrofuranyl, morpholinyl, and pyrrolidinyl, etc.

As used herein, the term “C₆-C₁₀ aryl” refers to an aryl with 6-10 carbon atoms, such as phenyl or naphthyl, or similar groups.

As used herein, the term “5-12-membered heteroaryl” refers to a cyclic aromatic group having 5-12 atoms of which 1-3 atoms are heteroatoms selected from N, S and O. It can be in the form of a single ring or a fused ring. Specific examples can be pyridinyl, pyridazinyl, pyrimidinyl, pyrazinyl, triazinyl, pyrrolyl, pyrazolyl, imidazolyl, (1,2,3)-triazolyl and (1,2,4)-triazolyl, tetrazolyl, furanyl, thienyl, isoxazole, thiazolyl, oxazolyl, etc.

Unless otherwise specified as “substituted or unsubstituted”, any group of the present invention can be substituted by substituents selected from the group consisting of: halogen, nitrile, nitro, hydroxy, amino, C₁-C₆ alkyl-amino, C₁-C₆ alkyl, C₂-C₆ alkenyl, C₂-Coalkynyl, C₁-C₆alkoxy, halogenated C₁-C₆ alkyl, halogenated C₂-C₆ alkenyl, halogenated C₂-Coalkynyl, halogenated C¹-C₆alkoxy, allyl, benzyl, C₆-C₁₀ aryl, C₁-C₆ alkoxy-C₁-C₆ alkyl, C₁-C₆alkoxy-carbonyl, phenoxycarbonyl, C₂-Coalkynyl-carbonyl, C₂-C₆ alkenyl-carbonyl, C₃-C₆cycloalkyl-carbonyl, C₁-C₆ alkyl-sulfonyl, etc.

As used herein, “halogen” or “halogen atom” refers to F, Cl, Br, and I. Preferably, halogen or halogen atomis selected from F, Cl, and Br. “Halogenated” refers to being replaced by atoms selected from F, Cl, Br, and I.

Unless otherwise specified, the structural formula described in the present invention is intended to include all isomeric forms (such as enantiomer, diastereomer and geometric isomers (or conformational isomerism)): for example, R and S configurations with asymmetric centers, (Z) and (E) isomers of double bonds, etc. Therefore, a single stereochemistry isomer of the compound of the invention or a mixture of its enantiomer, diastereomer or geometric isomers (or conformational isomer) all belong to the scope of the invention.

As used herein, the term “tautomer” means that structural isomers with different energies can cross the low energy barrier and transform into each other. For example, proton tautomer (i.e. prototropic change) includes tautomerism through prototropic change, such as 1H-indazole and 2H-indazole. The valence tautomer involves tautomerism by recombination of some bonding electrons.

As used herein, the term “solvate” refers to a complex formed by the coordination of the compound of the present invention with solvent molecule in a specific ratio.

As used herein, the term “hydrate” refers to a complex formed by the coordination of the compound of the present invention with water.

### The main advantages of the present invention include:

(1) The synthetic method of the present invention is simple to operate, the conditions are mild, the environmental pollution is low, and the raw materials are convenient to obtain.

(2) The synthetic method of the present invention has a high and stable yield and is suitable for industrial production.

The present invention is further described below in conjunction with specific embodiments. It is to be understood that these examples are intended to illustrate the invention only and not to limit the scope of the invention. The experimental methods in the following examples that do not specify specific conditions are usually based on conventional conditions or conditions recommended by the manufacturer. Unless otherwise specified, percentages and parts are percentages by weight and parts by weight.

Unless otherwise defined, all professional and scientific terms used herein have the same meanings as commonly understood by those skilled in the art. In addition, any methods and materials similar or equivalent to those described can be applied to the method of the present invention. The preferred embodiments and materials described herein are for exemplary purposes only.

The experimental materials and reagents used in the following examples are commercially available unless otherwise specified.

### EXAMPLE

### Example 1 Preparation of Benzyl 4-fluoro-4-(((1S,2R-2-phenylcyclopropyl)amino)methyl)piperidin-1-carboxylate hydrochloride (H1)

Synthesis route:

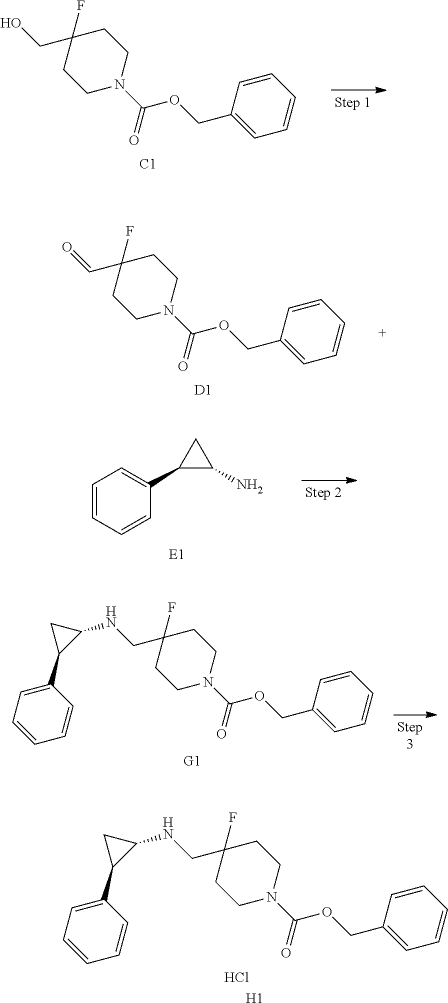

Preparation of D1:

Oxalyl chloride (57.5 mL, 680.2 mmol, 2 eq) was dissolved in anhydrous dichloromethane (1.03 L). The mixture was cooled down to −78 ° C., and added with anhydrous dimethylsulfoxide (72.4 mL, 1.022 mol, 3 eq) at a temperature no higher than −60° C. The reaction was then carried out at −78° C. for 0.5 hr followed by the addition of a solution of compound C1 (91.1 g, 340.7 mmol, 1 eq) in dichloromethane (250 mL).The reaction was reacted at −78° C. for 1.5 hours followed by the addition of triethylamine (235 mL, 1.703 mol, 5 eq), then continued to react at −78° C. for 10 minutes, then raised up to room temperature and reacted for 0.5 hours. The reaction solution was poured into 2.5 L of water for extraction; the aqueous phase was extracted with dichloromethane (300 mL×2);the organic phase was collected, washed with saturated saline (0.5 L×2), dried with anhydrous sodium sulfate, spun-dried and subjected to column chromatography with PE: EA=2:1 to give 77.7 g of yellow oily target compound D1, yield: 86%. LC-MS (ESI): m/z [M+H]⁺=266.

Preparation of G1:

Compound D1 (77 g, 290.3 mmol, 1 eq), (1S,2R)-2-phenylcyclopropylamine (38.7 g, 290.3 mmol, 1 eq) and formic acid (16.6 mL, 290.3 mmol, 1 eq) were dissolved in 500 mL of dichloromethane, and the mixture was evacuated and replaced with nitrogen for three times, and heated to reflux for 15 min. The reaction was then cooled to room temperature, added in batches with sodium cyanoborohydride (36.5 g, 580.6 mmol, 2 eq), and stirred at room temperature for 12 h. The reaction was quenched by slowly adding 300 mL of saturated sodium bicarbonate solution, and extracted with dichloromethane (300 mL×2). The organic phases were combined, then washed with saturated saline (0.5 L×2), dried with anhydrous sodium sulfate, spun-dried, and subjected to column chromatography with DCM: MeOH=20:1 to obtain 99 g of colourless oily target compound G1, yield: 89%.LC-MS (ESI): m/z [M+H]⁺=383.

Preparation of H1:

To a solution of Compound G1 (95 g, 248 mmol, 1 eq) in 1,4-dioxane (0.5 L) was addeddropwise 4 N HCl dioxane solution (63.2 mL, 253 mmol, 1.02 eq) under stirring and stirred overnight at room temperature. Then the mixture was spun-dried and re-dissolved in 100 mL of methanol, and then added with 2 L of ethyl acetate under stirring to precipitate white solid. After filteration, the white solid was washed with ethyl acetate and dried to give 96 g of white solid target compound H1. Yield: 92%. LC-MS (ESI): m/z [M+H]⁺=383.

### Example 2 3-Chlorobenzyl-4-fluoro-4-(((1S, 2R-2-phenylcyclopropyl) amino) methyl) piperidin-1-carboxylate hydrochloride (H2)

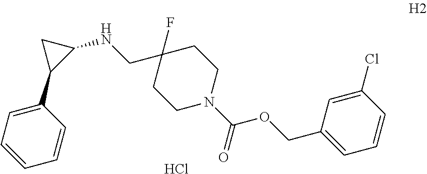 [SMILES: O=C(OCc1cccc(Cl)c1)N1CCC(F)(CN[C@H]2C[C@@H]2c2ccccc2)CC1 ; Cl]

By referring to the method for synthesizing H1, compound H2 was prepared by replacing the benzyl in the starting material with 3-chlorobenzyl. Total yield: 72%. LC-MS (ESI): m/z [M+H]⁺=417.

### Example 3 Furan-2-ylmethyl-4-fluoro-4-(((1S,2R-2-phenylcyclopropyl)amino)methyl)piperidin-1-carboxylate hydrochloride (H3)

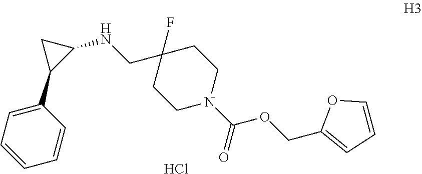 [SMILES: O=C(OCc1ccco1)N1CCC(F)(CN[C@H]2C[C@@H]2c2ccccc2)CC1 ; Cl]

By referring to the method for synthesizing H1, compound H3 was prepared by replacing the benzyl in the starting material with furan-2-ylmethyl. Total yield: 58%. LC-MS (ESI): m/z [M+H]⁺=373.

### Example 4 Preparation of Benzyl 4-fluoro-4-(((1S,2R-2-phenylcyclopropyl)amino)methyl)piperidin-1-carboxylate p-toluenesulfonate (H4)

Preparation route of H4:

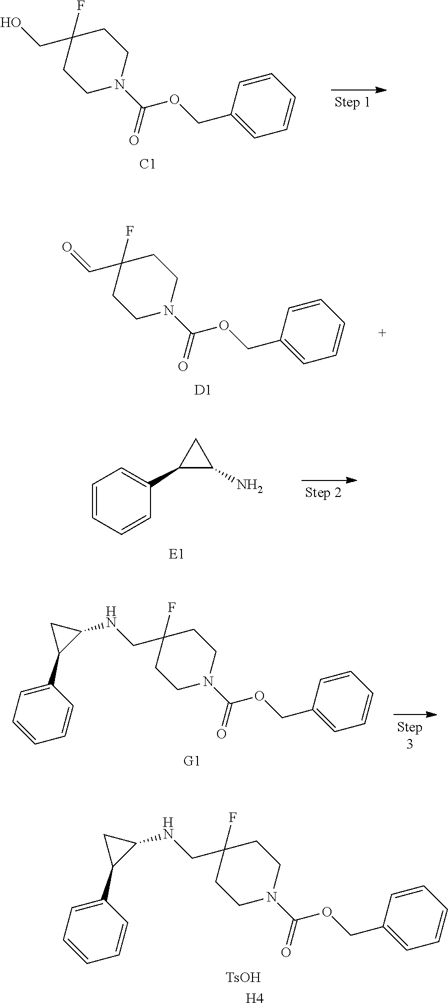

The synthetic route of G1 is as the above mentioned preparing route of H1.

Preparation of H4:

To a solution of Compound G1 (50 g, 131 mmol, 1 eq) in 1,4-dioxane (0.5 L) was added dropwise p-toluenesulfonic acid (23 g, 131 mmol, 1 eq) dioxane (100 mL) solution under stirring, and stirred at room temperature overnight. Then the mixture was spun-dried and re-dissolved in 300 mL of water/acetone (1/2, V/V), heated to 55° C. and stirred to dissolve clear, then cooled down to precipitate white solid. After filtration, the white solid was washed with 50 mL of water/acetone (1/2, V/V), and dried to give 60 g of white solid target compound H4. Yield: 82%.LC-MS(ESI): m/z [M+H]⁺=383.

### Example 5 Preparation of Cyclohexylmethyl-4-fluoro-4-(((1S,2R-2-phenylcyclopropyl)amino)methyl)piperidin-1-carboxylate p-toluenesulfonate (H5)

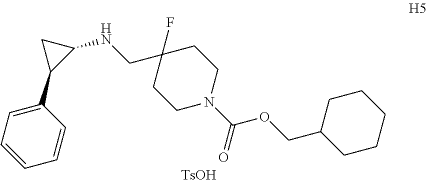

According to the method for synthesizing H4, compound H5 was prepared by replacing the benzyl in the starting material with cyclohexyl-methyl. Total yield: 67%. LC-MS (ESI): m/z [M+H]⁺=389.

### Example 6 Preparation of Pyridin-4-ylmethyl-4-fluoro-4-(((1S,2R-2-phenylcyclopropyl)amino)methyl)piperidin-1-carboxylate p-toluenesulfonate (H6)

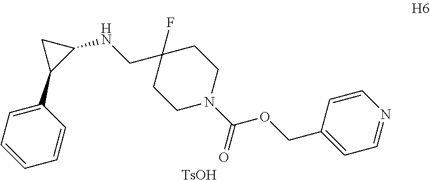

According to the method for synthesizing H4, compound H6 was prepared by replacing the benzyl in the starting material with pyridin-4-ylmethyl. Total yield: 45%. LC-MS (ESI): m/z [M+H]⁺=384.

### Example 7 Preparation of Thiophen-2-ylmethyl-4-fluoro-4-(((1S,2R-2-phenylcyclopropyl)amino)methyl)piperidin-1-carboxylate p-toluenesulfonate (H7)

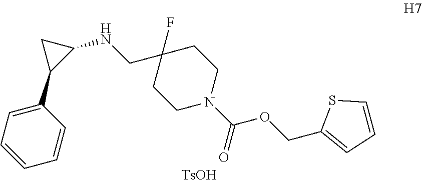

According to the method for synthesizing H4, compound H6 was prepared by replacing the benzyl in the starting material with thiophen-2-yl-methyl. Total yield: 46%. LC-MS (ESI): m/z [M+H]⁺=389.

### Example 8 Preparation of Benzyl 4-fluoro-4-(((1R,2S-2-phenylcyclopropyl)amino)methyl)piperidin-1-carboxylate hydrochloride (H8)

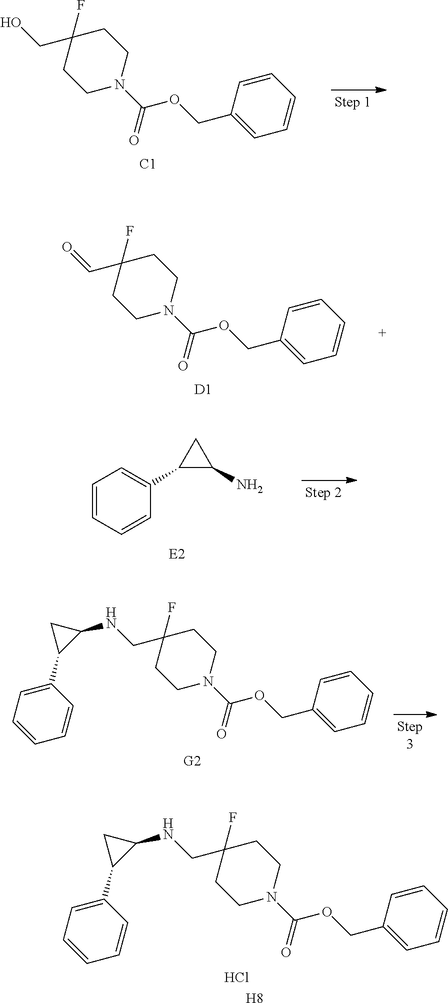

According to the method for synthesizing H1, compound H8 was prepared by replacing El with its enantiomer E2.

Preparation of G2:

Compound D1 (50 g, 188.5 mmol, 1 eq), (1R,2S)-2-phenylcyclopropylamine (25.1 g, 188.5 mmol, 1 eq) and formic acid (10.8 mL, 188.5 mmol, 1 eq) were dissolved in 300 mL of dichloromethane, and the mixture was evacuated and replaced with nitrogen for three times, and heated to reflux for 15 min. The reaction was then cooled to room temperature followed by the addition of sodium cyanoborohydride (23.7 g, 580.6 mmol, 2 eq) in batches, and stirred at room temperature for 12 h. The reaction was quenched by slowly adding 150 mL of saturated sodium bicarbonate solution, and extracted with dichloromethane (150 mL×2).The organic phases were combined, then washed with saturated saline (0.5 L×2), dried over anhydrous sodium sulfate, spun-dried, and subjected to column chromatography with DCM: MeOH=20:1 to obtain 60.5 g of colourless oily target compound G1, yield: 84%. LC-MS (ESI): m/z [M+H]⁺=383.

Preparation of H8:

To a solution of Compound G2 (55 g, 144 mmol, 1 eq) in 1,4-dioxane (0.4 L) was added dropwise 4 N HCldioxane solution (36.7 mL, 147 mmol, 1.02 eq) under stirring and stirred overnight at room temperature. Then the mixture was spun-dried and re-dissolved in 80 mL of methanol, and added with 1.6 L of ethyl acetate under stirring to precipitate white solids. After filteration, the white solid was washed with ethyl acetate and dried to give 54 g of white solid target compound H8. Yield: 90%. LC-MS (ESI): m/z [M+H]⁺=383.

### Example 9 Preparation of 4-fluorobenzyl-4-fluoro-4-(((1R,2S-2-phenylcyclopropyl)amino)methyl)piperidin-1-carboxylate hydrochloride (H9)

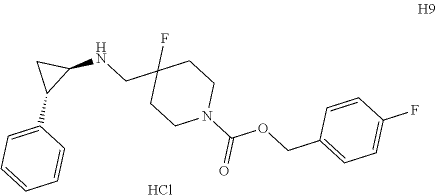 [SMILES: O=C(OCc1ccc(F)cc1)N1CCC(F)(CN[C@@H]2C[C@H]2c2ccccc2)CC1 ; Cl]

According to the method for synthesizing H8, compound H9 was prepared by replacing the benzyl in the starting material with 4-fluorobenzyl. Total yield: 52%. LC-MS (ESI): m/z [M+H]⁺=401.

### Example 10 Preparation of Benzyl 4-methoxybenzyl-4-fluoro-4-(((1R,2S-2-phenylcyclopropyl)amino)methyl)piperidin-1-carboxylate hydrochloride (H10)

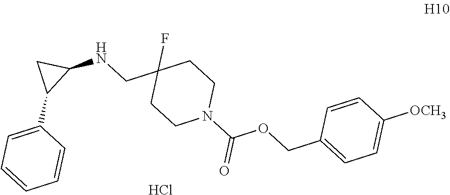 [SMILES: COc1ccc(COC(=O)N2CCC(F)(CN[C@@H]3C[C@H]3c3ccccc3)CC2)cc1 ; Cl]

According to the method for synthesizing H8, compound H9 was prepared by replacing the benzyl in the starting material with 4-methoxybenzyl. Total yield: 57%. LC-MS (ESI): m/z [M+H]⁺=413.

### Example 11 Preparation of Piperidin-4-methyl-4-fluoro-4-(((1S,2R-2-phenylcyclopropyl)amino)methyl)piperidin-1-carboxylate hydrochloride (H11)

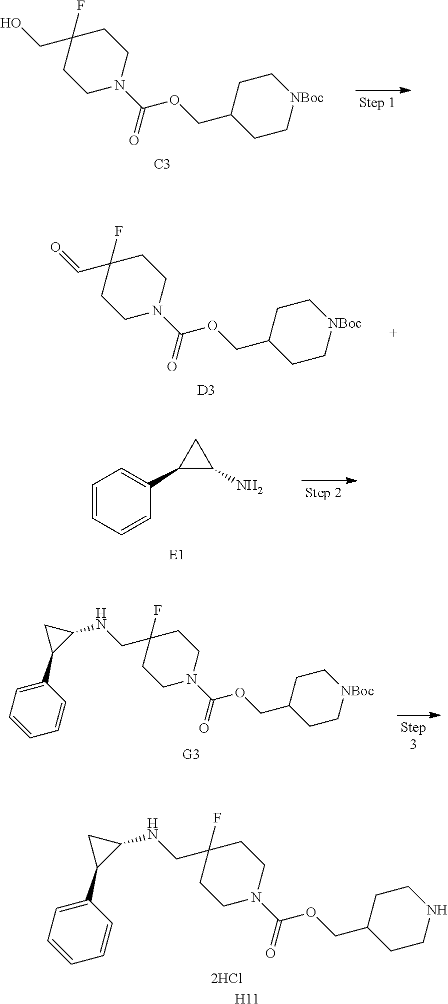

Preparation of D3:

Oxalyl chloride (45.1 mL, 534 mmol, 2 eq) was dissolved in anhydrous dichloromethane (1 L), and the mixture was cooled down to −78° C., and added with anhydrous dimethylsulfoxide (56.7 mL, 0.801 mol, 3 eq) at a temperature no higher than −60° C. The reaction was then carried out at −78° C. for 0.5 hr, after which a solution of compound C₃ (100 g, 267 mmol, 1 eq) in dichloromethane (250 mL) was added. After reacting at −78° C. for 1.5 hours, the reaction was added with triethylamine (184 mL, 1.335 mol, 5 eq) and continued at −78° C. for 10 minutes, then raised up to room temperature and reacted for 0.5 hours. The reaction solution was poured into 2.5 L of water for extraction; the aqueous phase was extracted with dichloromethane (300 mL×2), and the organic phase was collected, washed with saturated saline (0.5 L×2), dried with anhydrous sodium sulfate, spun-dried and subjected to column chromatography with PE : EA=2:1 to give 87.6 g of yellow oily target compound D3, yield: 88%. LC-MS (ESI): m/z [M+H]⁺=373.

Preparation of G3:

Compound D3 (80 g, 301.6 mmol, 1 eq), (1S,2R)-2-phenylcyclopropylamine (40.2 g, 301.6 mmol, 1 eq) and formic acid (17.2 mL, 301.6 mmol, 1 eq) were dissolved in 500 mL of dichloromethane, and the mixture was evacuated and replaced with nitrogen for three times, and heated to reflux for 15 min. The reaction was then cooled to room temperature followed by the addition of sodium cyanoborohydride (37.9 g, 603.2 mmol, 2 eq) in batches, and then stirred at room temperature for 12 h. The reaction was quenched by slowly adding 310 mL of saturated sodium bicarbonate solution, and extracted with dichloromethane (300 mL×2).The organic phases were combined, then washed with saturated saline (0.5 L×2), dried over anhydrous sodium sulfate, spun-dried, and subjected to column chromatography with DCM: MeOH=20:1 to obtain 123 g of colourless oily target compound G3, yield: 83%. LC-MS (ESI): m/z [M+H]⁺=490.

Preparation of H11:

To a solution of Compound G3 (110 g, 287.2 mmol, 1 eq) in 1,4-dioxane (0.5 L) was added dropwise 4 N HCldioxane solution (287 mL, 1148.8 mmol, 4.0 eq) under stirring and stirred overnight at room temperature. Then the mixture was spun-dried, re-dissolved in 100 mL of methanol, and added with 2 L of ethyl acetateunder stirring to precipitate white solid. After filtration, the white solid was washed with ethyl acetate and dried to give 123 g of white solid target compound H11. Yield: 93%. LC-MS (ESI): m/z [M+H]⁺=390.

### Example 12 Preparation of Piperidin-4-methyl-4-fluoro-4-(((1S,2R-2-phenylcyclopropyl)amino)methyl)piperidin-1-carboxylate p-toluenesulfonate (H12)

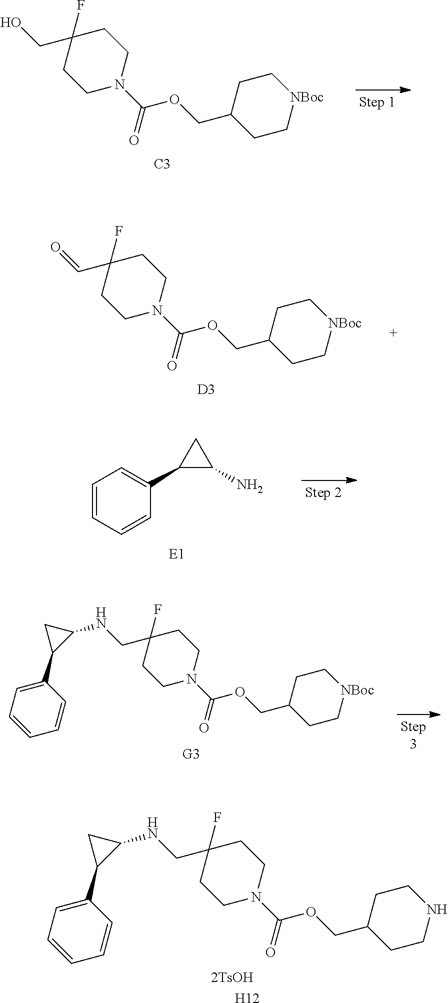

Preparation of H12:

To a solution of Compound G3 (100 g, 261.1 mmol, 1 eq) in 1,4-dioxane (1 L) was addeddropwise p-toluenesulfonic acid (180 g, 1044.4 mmmol, 4 eq) dioxane (200 mL) solution under stirring, and stirred at room temperature overnight. Then the mixture was spun-dried and re-dissolved in 600 mL of water/acetone (1/2, V/V), heated to 55° C. and stirred to dissolve clear, and cooled down to precipitate white solid. After filtration, the white solid was washed with 100 mL of water/acetone (1/2, V/V), and dried to give 153 g of white solid target compound H12. Yield: 80%. LC-MS (ESI): m/z [M+H]⁺=390.

### Example 13 Preparation of piperidin-4-methyl-4-fluoro-4-(((1R,2S-2-phenylcyclopropyl)amino)methyl)piperidin-1-carboxylate hydrochloride (H13)

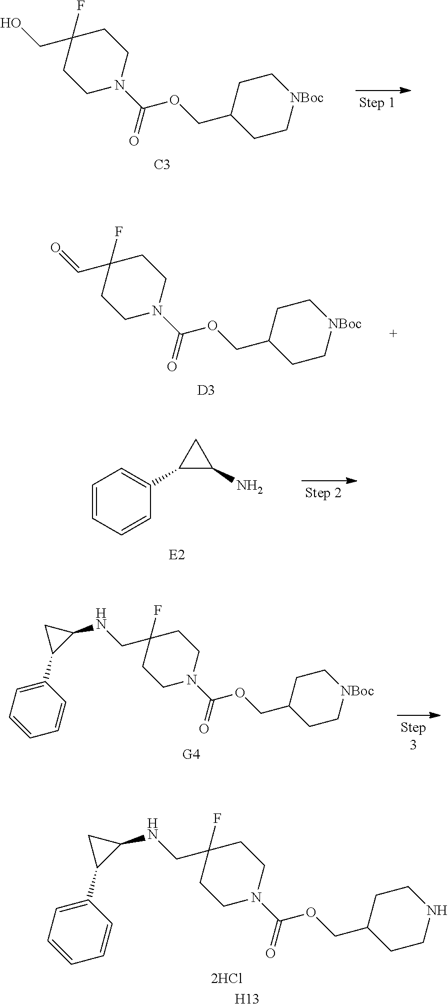

According to the method for synthesizing H1, compound H13 was prepared by replacing E1 with its enantiomer E2.

Preparation of G4:

Compound D3 (50 g, 188.5 mmol, 1 eq), (1R,2S)-2-phenylcyclopropylamine (25.1 g, 301.6 mmol, 1 eq) and formic acid (10.8 mL, 188.5 mmol, 1 eq) were dissolved in 300 mL of dichloromethane, and the mixture was evacuated and replaced with nitrogen for three times, and heated to reflux for 15 min. The reaction was then cooled to room temperature, added in batches with sodium cyanoborohydride (23.7 g, 377 mmol, 2 eq), and stirred at room temperature for 12 h. The reaction was quenched by slowly adding 180 mL of saturated sodium bicarbonate solution, and extracted with dichloromethane (180 mL×2). The organic phases were combined, then washed with saturated saline (0.3 L×2), dried over anhydrous sodium sulfate, spun-dried, and subjected to column chromatography with DCM: MeOH=20:1 to obtain 78 g of colourless oily target compound G4, yield: 85%. LC-MS (ESI): m/z [M+H]⁺=490.

Preparation of H13:

To a solution of Compound G4 (50 g, 130.5 mmol, 1 eq) in 1,4-dioxane (0.25 L) was added dropwise 4 N HCldioxane solution (131 mL, 522 mmol, 4.0 eq) under stirring and stirred overnight at room temperature. Then the mixture was spun-dried, re-dissolved in 60 mL of methanol, and added with 1 L of ethyl acetate under stirring to precipitate white solid. After filtration, the white solid was washed with ethyl acetate and dried to give 52 g of white solid target compound H13. Yield: 87%. LC-MS (ESI): m/z [M+H]⁺=390.

### Example 14 Preparation of Piperidin-4-methyl-4-fluoro-4-(((1R,2S-2-phenylcyclopropyl)amino)methyl)piperidin-1-carboxylate p-toluenesulfonate (H14)

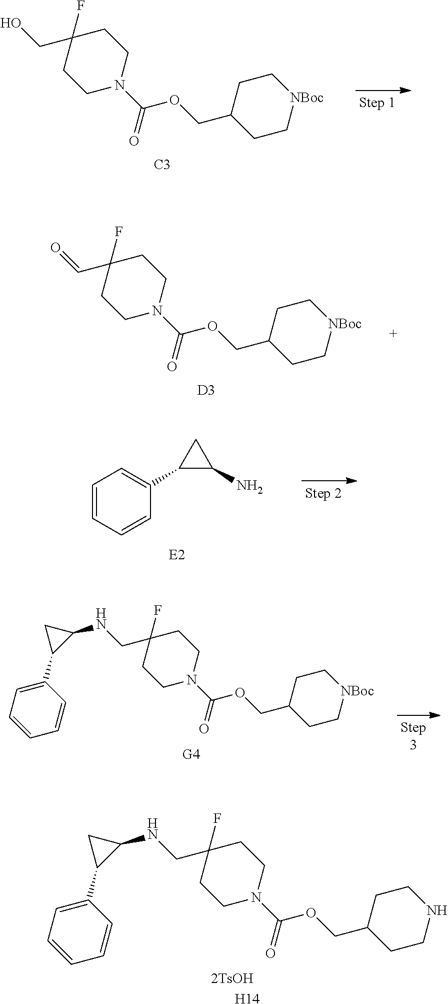

### Preparation of H14:

To a solution of Compound G4 (50 g, 130 mmol, 1 eq) in 1,4-dioxane (0.5 L) was added dropwise p-toluenesulfonic acid (90 g, 522 mmmol, 1 eq) dioxane (100 mL) solution under stirring, and stirred at room temperature overnight. Then the mixture was spun-dried and re-dissolved in 300 ml of water/acetone (1/2, V/V), heated to 55° C. and stirred to dissolve clear, and cooled down to precipitate white solid. After filtration, the white solid was washed with 50 ml of water/acetone (1/2, V/V), and dried to give 80 g of white solid target compound H14. Yield: 84%. LC-MS (ESI): m/z [M+H]⁺=390.

### Example 15 Preparation of Piperidin-4-methyl-4-fluoro-4-(((1S,2R-2-phenylcyclopropyl)amino)methyl)piperidin-1-carboxylate p-toluenesulfonate (K1)

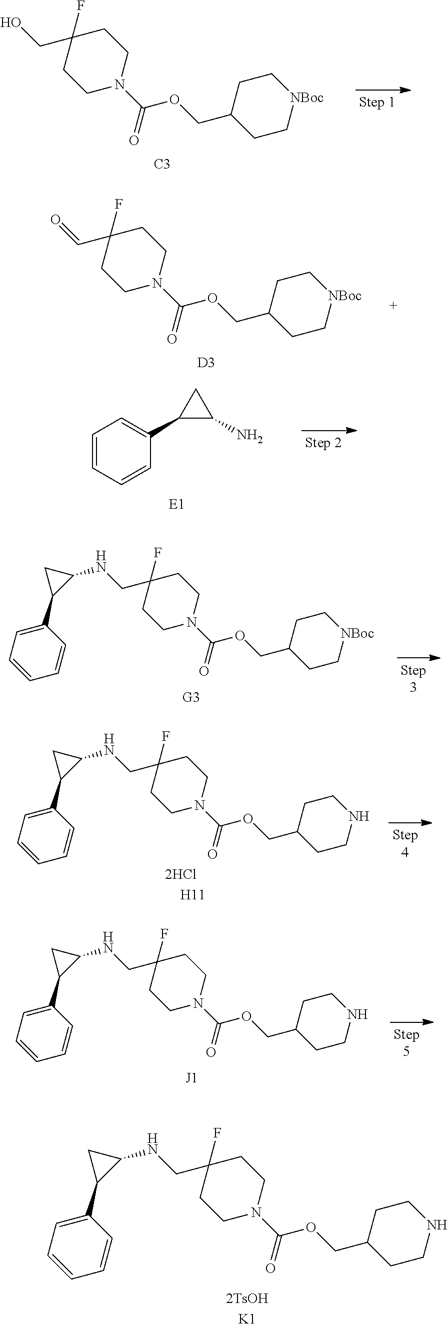

Preparation of J1:

Compound H11 (50 g, 108 mmol) was dissolved in saturated sodium hydroxide aqueous solution (100 mL). After stirring for 30 min, the mixture was extracted with dichloromethane (3*1000 mL), and the organic phases were combined, dried over anhydrous sodium sulfate, and concentrated to give 30.5 g of crude product, which was directly used in the next step. Yield: 72.4%. LC-MS (ESI): m/z [M+H]⁺=390.

Preparation of K1:

Compound J1 (30 g, 77.0 mmol) was placed in a reactor and was configured as a 0.12 M solution by adding a quantitative amount of acetonitrile(about 321 mL of acetonitrile), and then 0.12 M configured p-toluenesulfonic acid solution (6.63 g, 38.51 mmol, 321 mL) was added. The reaction solution was stirred at 60° C. for 1 h, then cooled down to room temperature and stood overnight. The precipitated solid was filtered and washed three times with a small amount of acetonitrile. The obtained solid was then dissolved in 100 mL of ethyl acetate, stirred overnight and filtered to obtain 26.8 g of white solid target compound K1. Yield: 62%. LC-MS (ESI): m/z [M+H]⁺=390.

### Example 16 Preparation of Piperidin-4-methyl-4-fluoro-4-(((1R,2S-2-phenylcyclopropyl)amino)methyl)piperidin-1-carboxylate p-toluenesulfonate (K2)

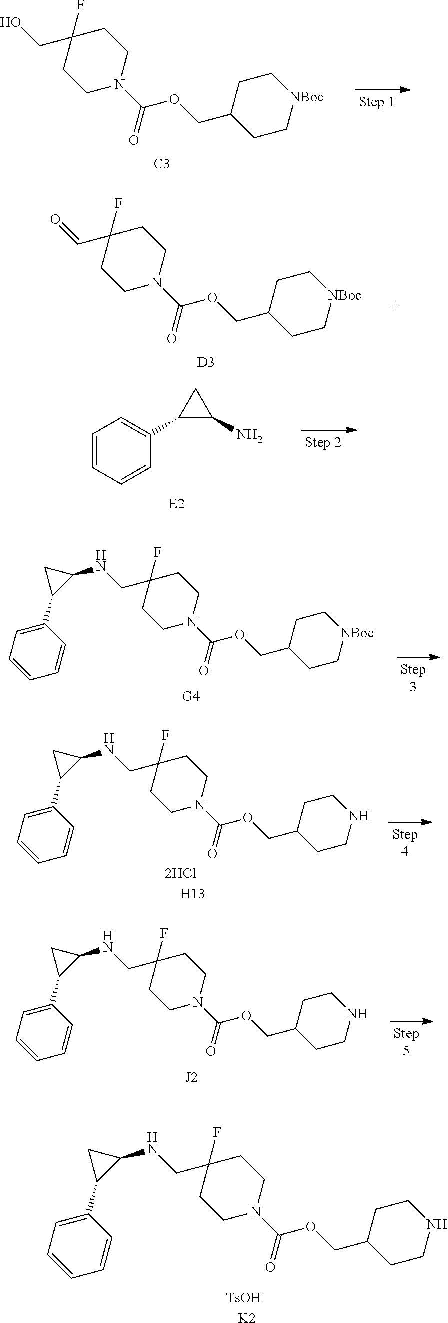

Preparation of J2:

Compound H13 (50 g, 108 mmol) was dissolved in saturated sodium hydroxide aqueous solution (100 mL), and stirred for 30 min. The mixture was then extracted with dichloromethane (3*1000 mL), and the organic phases were combined, dried with anhydrous sodium sulfate, and concentrated to give 32 g of crude product, which was directly used in the next step. Yield: 76%. LC-MS (ESI): m/z [M+H]⁺=390.

Preparation of K2:

Compound J2 (30 g, 77.0 mmol) was placed in a reactor and was configured as a 0.12 M solution by adding a quantitative amount of acetonitrile (about 321 mL of acetonitrile), and then 0.12 M configured p-toluenesulfonic acid solution (6.63 g, 38.51 mmol, 321 mL) was added. The reaction solution was stirred at 60° C. for 1 h, then cooled down to room temperature and stood overnight. The precipitated solid was filtered and washed three times with a small amount of acetonitrile. The obtained solid was then dissolved in 100 mL of ethyl acetate, stirred overnight and filtered to obtain 28 g of white solid target compound K2. Yield: 65%. LC-MS (ESI): m/z [M+H]⁺=390.

### Example 17 Preparation of Benzyl 4-fluoro-4-(((1S,2R-2-phenylcyclopropyl)amino)methyl)piperidin-1-carboxylate sulfate (K3)

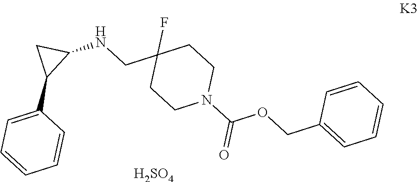 [SMILES: O=C(OCc1ccccc1)N1CCC(F)(CN[C@H]2C[C@@H]2c2ccccc2)CC1 ; "H2SO4"]

Compound G1 (30 g, 78.44 mmol, 1 eq) was dissolved in acetonitrile (0.65 L), and 0.12 M configured acetonitrile solution of sulphuric acid (0.33 L) was added. The reaction solution was stirred at 60° C. for 1 h, then cooled down to room temperature and stood overnight. The precipitated solid was filtered and the filter cake was washed 3 times with a small amount of acetonitrile after filtration. 33 g of white solid target compound K3 was obtained. Yield: 88%. LC-MS (ESI): m/z [M+H]⁺=383.

### Example 18 Preparation of Benzyl 4-fluoro-4-(((1R,2S-2-phenylcyclopropyl)amino)methyl)piperidin-1-carboxylate sulfate (K4)

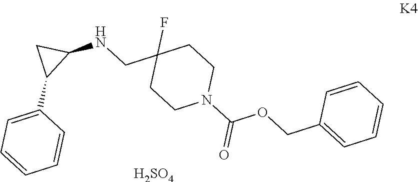 [SMILES: O=C(OCc1ccccc1)N1CCC(F)(CN[C@@H]2C[C@H]2c2ccccc2)CC1 ; "H2SO4"]

According to the method for synthesizing K3, compound K4 was prepared by replacing G1 with its enantiomer G2. Yield: 79%. LC-MS (ESI): m/z [M+H]⁺=383.

### Example 19 Preparation of 3-Chlorobenzyl-4-fluoro-4-(((1S,2R-2-phenylcyclopropyl)amino)methyl)piperidin-1-carboxylate sulfate (K5)

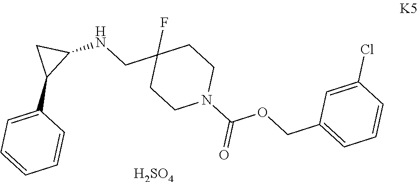 [SMILES: O=C(OCc1cccc(Cl)c1)N1CCC(F)(CN[C@H]2C[C@@H]2c2ccccc2)CC1 ; "H2SO4"]

According to the method for synthesizing K3, compound K5 was prepared by replacing the benzyl in the starting material with 3-chlorobenzyl. Total yield: 70%. LC-MS (ESI): m/z [M+H]⁺=417.

### Example 20 Preparation of 4-Methoxybenzyl-4-fluoro-4-(((1R,2S-2-phenylcyclopropyl)amino)methyl)piperidin-1-carboxylate sulfate (K6)

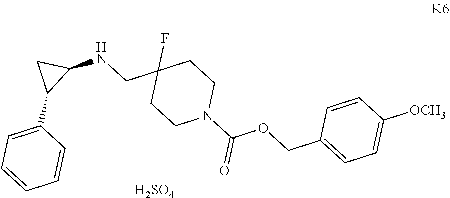 [SMILES: COc1ccc(COC(=O)N2CCC(F)(CN[C@@H]3C[C@H]3c3ccccc3)CC2)cc1 ; "H2SO4"]

According to the method for synthesizing K3, compound K6 was prepared by using the prepared free base H10 as a raw material. Total yield: 85%. LC-MS (ESI): m/z [M+H]⁺=413.

### Example 21 Preparation of Piperidin-4-methyl-4-fluoro-4-(((1S,2R-2-phenylcyclopropyl)amino)methyl)piperidin-1-carboxylate sulfate (K7)

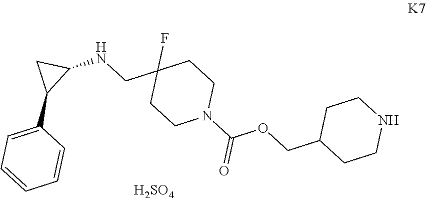 [SMILES: O=C(OCC1CCNCC1)N1CCC(F)(CN[C@H]2C[C@@H]2c2ccccc2)CC1 ; "H2SO4"]

According to the method for synthesizing K3, compound K7 was prepared by using J1 as a raw material. Total yield: 83%. LC-MS (ESI): m/z [M+H]⁺=390.

### Example 22 Preparation of Piperidin-4-methyl-4-fluoro-4-(((1R,2S-2-phenylcyclopropyl)amino)methyl)piperidin-1-carboxylate sulfate (K8)

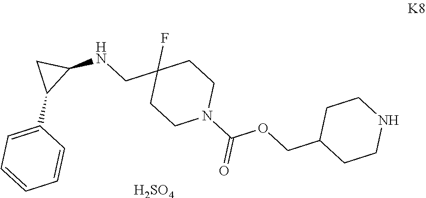 [SMILES: O=C(OCC1CCNCC1)N1CCC(F)(CN[C@@H]2C[C@H]2c2ccccc2)CC1 ; "H2SO4"]

According to the method for synthesizing K3, compound K8 was prepared by using J2 as a raw material. Total yield: 90%. LC-MS (ESI): m/z [M+H]⁺=390.

### Example 23 Preparation of Benzyl 4-fluoro-4-(((1S,2R-2-phenylcyclopropyl)amino)methyl)piperidin-1-carboxylate oxalate (K9)

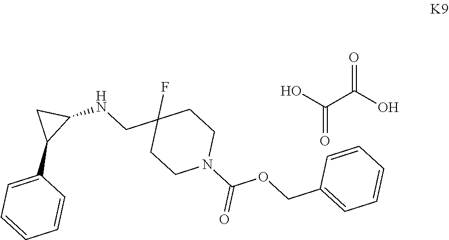

Compound G1 (50 g, 130.73 mmol, 1 eq) was dissolved in acetonitrile (1.1 L), and 0.12 M acetonitrile solution of oxalic acid (11.8 g, 130.73 mmol, 1.1 L) was added. The reaction solution was stirred at 60° C. for 1 h, then cooled down to room temperature and stood overnight. The precipitated solid was filtered and the filter cake was washed 3 times with a small amount of acetonitrile after filtration. 55 g of white solid target compound K9 was obtained. Yield: 89%. LC-MS (ESI): m/z [M+H]⁺=383.

### Example 24 Preparation of Benzyl 4-fluoro-4-(((1R,2S-2-phenylcyclopropyl)amino)methyl)piperidin-1-carboxylate oxalate (K10)

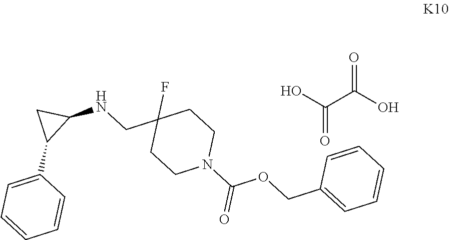

According to the method for synthesizing K9, compound K10 was prepared by replacing G1 with its enantiomer G2. Yield: 84%. LC-MS (ESI): m/z [M+H]⁺=383.

### Example 25 Preparation of Thiophen-2-ylmethyl-4-fluoro-4-(((1S,2R-2-phenylcyclopropyl)amino)methyl)piperidin-1-carboxylate oxalate (K11)

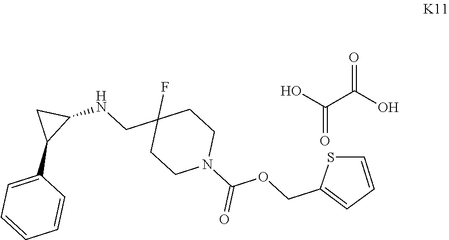

According to the method for synthesizing K9, compound K11 was prepared by replacing the benzyl in the starting material with thiophen-2-ylmethyl. Yield: 72%. LC-MS (ESI): m/z [M+H]⁺=389.

### Example 26 Preparation of Piperidin-4-methyl-4-fluoro-4-(((1S,2R-2-phenylcyclopropyl)amino)methyl)piperidin-1-carboxylate oxalate (K12)

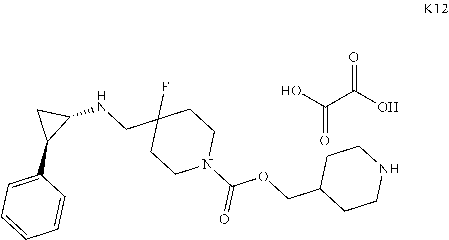

According to the method for synthesizing K9, compound K12 was prepared by using J1 as a raw material. Total yield: 81%. LC-MS (ESI): m/z [M+H]⁺=390.

### Example 27 Preparation of piperidin-4-methyl-4-fluoro-4-(((1R,2S-2-phenylcyclopropyl)amino)methyl)piperidin-1-carboxylate oxalate (K13)

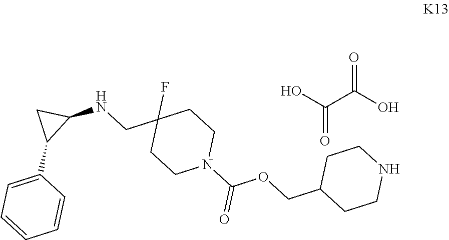

According to the method for synthesizing K9, compound K13 was prepared by using J2 as a raw material. Total yield: 87%. LC-MS (ESI): m/z [M+H]⁺=390.

All documents referred to in the present invention are incorporated by reference herein as if each document is individually incorporated by reference. Further, it should be understood that upon reading the above teaching of the present invention, various alterations or modifications may be made to the present invention by those skilled in the art, and those equivalents also fall within the scope defined by the appended claims of the present application.

1. A method for preparing a compound of formula I, or a pharmaceutically acceptable salt thereof: 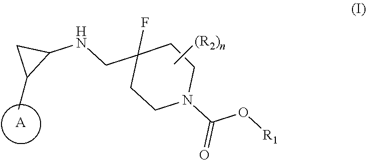
wherein,
ring A is selected from the group consisting of: substituted or unsubstituted C₆-C₁₀ aromatic ring, substituted or unsubstituted 5-12 membered heteroaromatic ring;
R₁ is selected from the group consisting of: —CH₂—R; wherein R is selected from the group consisting of: H, C₁-C₄ alkyl, substituted or unsubstituted C₆-C₁₀ aryl, substituted or unsubstituted C₃-C₈cycloalkyl, substituted or unsubstituted 5- to 10-membered heteroaryl, substituted or unsubstituted 4- to 10-membered heterocyclyl;
wherein the “substituted” refers to one or more hydrogen atoms on the group being substituted by groups selected from the group consisting of: halogen, C₁-C₄ alkyl, hydroxyl, carboxyl, C₁-C₄alkoxy, C₁-C₄haloalkyl, and amino-protecting group;
wherein the heteroaryl or heterocyclyl comprises 1, 2, or 3 heteroatoms selected from the group consisting of: N, O, and S; and wherein the amino-protecting group is selected from the group consisting of: Boc, SEM, Cbz, Fmoc, Alloc, Teoc, Tos, Tfa, PMB and Bn;
each R₂ is independently selected from the group consisting of: methyl;
n is 0, 1 or 2;
the method comprises steps: 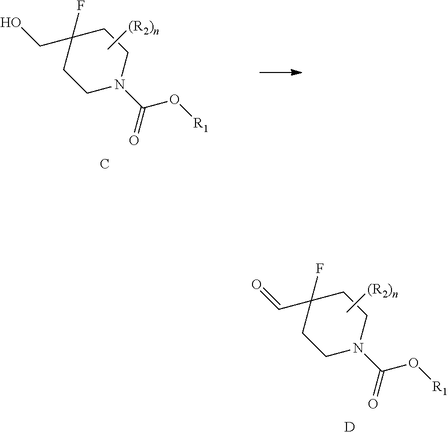
(1) In a solvent, converting compound C into compound D in the presence of an oxidizing agent;
wherein the oxidising agent is selected from the group consisting of: potassium chromate sulfate, pyridine chromium trioxide/pyridine, pyridine chromium trioxide, pyridine chlorochromic acid, oxalyl chloride/dimethylsulfoxide, pyridine sulfur trioxide/dimethylsulfoxide/triethylamine, Dess-Martin high iodine oxidiser, and iodosobenzene diacetate/TEMPO;
the solvent is selected from the group consisting of: C₁-C₄ halogenated hydrocarbon solvent, acetone, acetonitrile, tetrahydrofuran, ethyl acetate, toluene, dioxane, and combinations thereof; 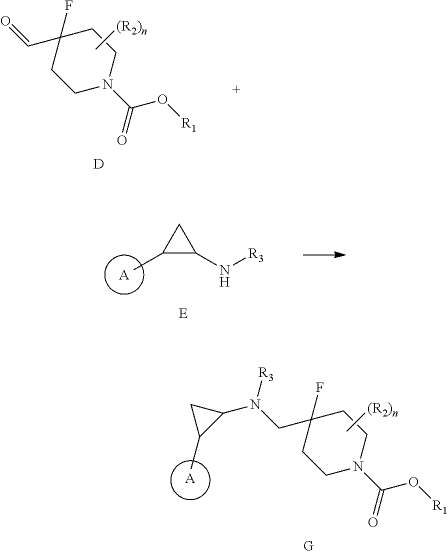
(2) Compound D and Compound E undergo reductive amination to produce Compound G.
In another preferred embodiment, step (2) is carried out in the presence of a reducing agent and an additive.
In another preferred embodiment, the reducing agent is selected from the group consisting of: sodium cyanoborohydride, sodium triacetoxyborohydride, sodium borohydride, and combinations thereof.
In another preferred embodiment, the additive is selected from the group consisting of: formic acid, acetic acid, and combinations thereof.
In another preferred embodiment, step (2) is carried out in a solvent selected from the group consisting of: C₁-C₄ halogenated hydrocarbon solvent, C₁-C₆ alcoholic solvent, tetrahydrofuran, 2-methyltetrahydrofuran, ethyl acetate, isopropyl acetate, and combinations thereof.
In another preferred embodiment, R is selected from the group consisting of:
substituted or unsubstituted phenyl, substituted or unsubstituted C₅-C₆cycloalkyl, substituted or unsubstituted 5-7-membered heteroaryl, and substituted or unsubstituted 5-6-membered heterocyclyl; wherein the “substituted” refers to one or more hydrogen atoms on the group being substituted by groups selected from the group consisting of: halogen, C₁-C₄ alkyl, hydroxyl, carboxyl, C₁-C₄alkoxy, C₁-C₄ haloalkyl, and amino-protecting group; the heteroaryl or heterocyclyl comprises 1, 2, or 3 heteroatoms selected from the group consisting of: N, O, and S; and the amino-protecting group is selected from the group consisting of: Boc, SEM, Cbz, Fmoc, Alloc, Teoc, Tos, Tfa, PMB, and Bn.
In another preferred embodiment, R₁ is selected from the group consisting of: ethyl, benzyl, phenyl, cyclohexylmethyl, 4-pyridinylmethyl, phenylethyl, 1H-indole-5-ylmethyl, 2-thienylmethyl, 2-furyl methyl, 4-fluorophenylmethyl, 4-chlorophenylmethyl, 4-bromophenylmethyl, 4-methoxyphenyl, 4-trifluoromethylphenylmethyl, 3,5-dimethoxyphenylmethyl, 4-carboxyphenylmethyl, cyclopentyl methyl, cyclobutylmethyl, 4-piperidylmethyl, 3-chlorophenylmethyl, 2-chlorophenylmethyl, and 4-tert butylphenylmethyl.
In another preferred embodiment, the compound of formula G is selected from the group consisting of:

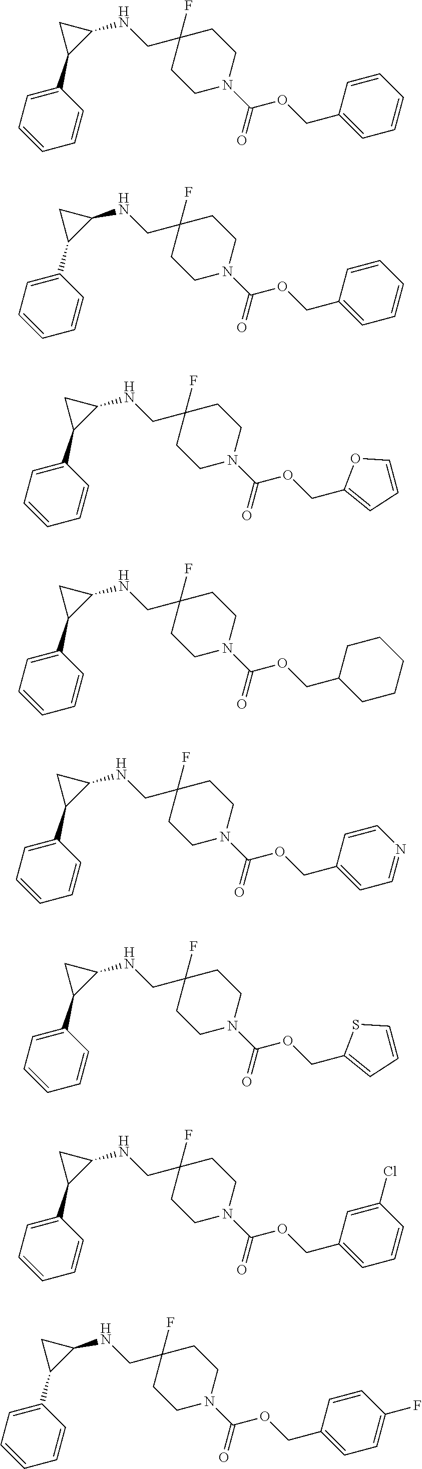

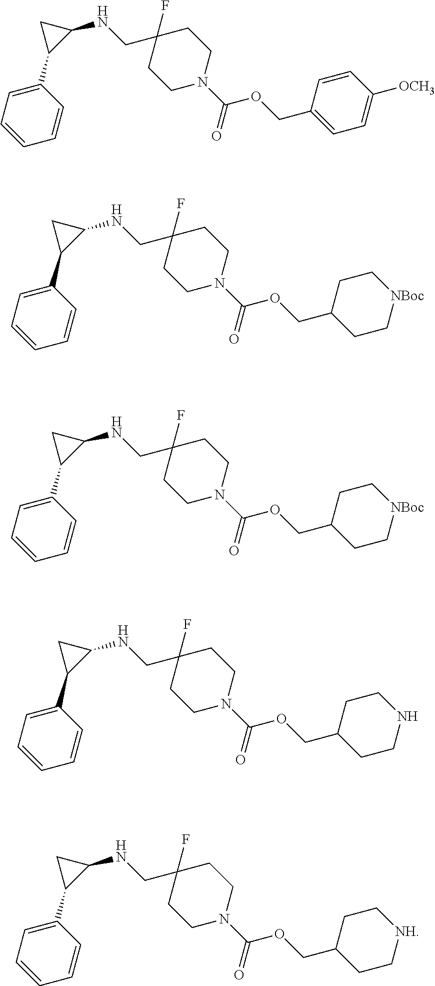 [SMILES: COc1ccc(COC(=O)N2CCC(F)(CN[C@@H]3C[C@H]3c3ccccc3)CC2)cc1 ; CC(C)(C)OC(=O)N1CCC(COC(=O)N2CCC(F)(CN[C@H]3C[C@@H]3c3ccccc3)CC2)CC1 ; CC(C)(C)OC(=O)N1CCC(COC(=O)N2CCC(F)(CN[C@@H]3C[C@H]3c3ccccc3)CC2)CC1 ; O=C(OCC1CCNCC1)N1CCC(F)(CN[C@H]2C[C@@H]2c2ccccc2)CC1 ; O=C(OCC1CCNCC1)N1CCC(F)(CN[C@@H]2C[C@H]2c2ccccc2)CC1]

2. The method according to claim 1, wherein in step (1), the oxidising agent is selected from the group consisting of: oxalyl chloride/dimethylsulfoxide, pyridine sulfur trioxide/dimethylsulfoxide/triethylamine, Dess-Martin oxidiser, and iodosobenzene diacetate/TEMPO.

3. The method according to claim 1, wherein in step (1), the solvent is a halogenated hydrocarbon solvent.

4. The method according to claim 1, wherein in step (1), the solvent is dichloromethane.

5. The method according to claim 1, wherein in step (1), the molar ratio of the compound of formula C to the oxidising agent is 1:1.0 -1:6.0.
In another preferred embodiment, the temperature of the reaction is −80˜40° C.
In another preferred embodiment, the time of the reaction is 4-48 hours.
In another preferred embodiment, the ratio V/W of the volume of the reaction solvent used to the weight of the substrate is 5-20 mL/g.

6. The method according to claim 1, wherein in step (1), the molar ratio of the compound of formula C to the oxidising agent is 1: 2.0 -1: 4.0.
In another preferred embodiment, the temperature of the reaction is −78-20° C.
In another preferred embodiment, the time of the reaction is 2-24 hours.

7. The method according to claim 1, wherein in step (2), the reducing agent is selected from the group consisting of: sodium cyanoborohydride, and sodium triacetoxyborohydride.

8. The method according to claim 1, wherein in step (2), the solvent is selected from the group consisting of: C₁-C₆ alcoholic solvent, and C₁-C₄ halogenated hydrocarbon solvent.

9. The method according to claim 1, wherein in step (2), the molar ratio of the compound of formula D to the reducing agent is 1:0.8 -1:2.0; and/or the molar ratio of the compound of formula D to the compound of formula E is 1:0.5 -1:2.0; and/or
the molar ratio of the compound of formula D to the additive is 1:0.5-1:2.0.
In another preferred embodiment, the temperature of the reaction is −10˜50° C.
In another preferred embodiment, the time of the reaction is 1 -48 hours.
In another preferred embodiment, the ratio V/W of the volume of the reaction solvent used to the weight of the substrate is 5 mL/g˜30 mL/g.

10. The method of claim 1, wherein the method further comprises steps: 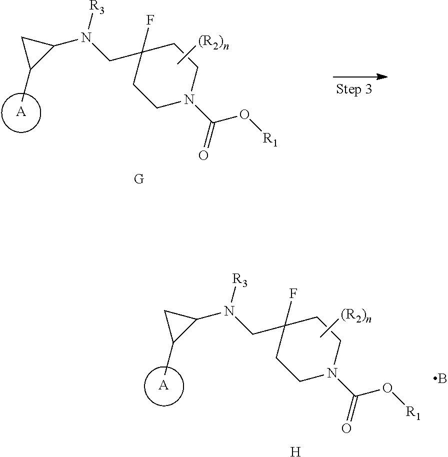
(3) Reacting compound G with an organic or inorganic acid to obtain compound H.
In another preferred embodiment, in step (3), the organic or inorganic acid is selected from the group consisting of: hydrochloric acid, hydrobromic acid, sulfuric acid, nitric acid, sulfamicacid, phosphoric acid, citric acid, tartaric acid, lactic acid, pyruvic acid, acetic acid, benzenesulfonic acid, p-toluenesulfonic acid, methanesulfonic acid, naphthalene sulfonic acid, ethanesulfonic acid, naphthalene disulfonic acid, maleic acid, malic acid, malonic acid, fumaric acid, succinic acid, propionic acid, oxalic acid, trifluoroacetic acid, stearic acid, pamoic acid, hydroxymaleic acid, phenylacetic acid, benzoic acid, salicylic acid, glutamic acid, ascorbic acid, p-aminobenzenesulfonic acid, 2-acetyloxybenzoic acid, hydroxyethanesulfonic acid, and combinations thereof.
In another preferred embodiment, in step (3), the solvent is selected from the group consisting of: methanol, ethanol, dimethyl sulfoxide, methyl tert-butyl ether, dioxane, acetone, acetonitrile, tetrahydrofuran, ethyl acetate, isopropyl acetate, and combinations thereof.
In another preferred embodiment, in step (3), the solvent is selected from the group consisting of: mixed solvent of C₁-C₆ alcoholic solvent/water, mixed solvent of acetonitrile/water, and mixed solvent of acetone/water.

11. The method according to claim 10, wherein in step (3), the molar ratio of the compound of formula G to the salt-forming reagent is 1:1.0˜5.0.
In another preferred embodiment, in step (3), the temperature of the reaction is preferably 20-100° C.
In another preferred embodiment, in step (3), the time of the reaction is preferably 2 -48 hours.
In another preferred embodiment, in step (3), the ratio (V/W)of the volume of the reaction solvent used to the weight of the substrate is 5 mL/g˜30 mL/g.

12. A method for preparing intermediate compound of formula D: 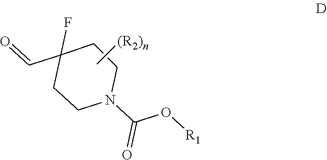
wherein,
R₁ is selected from the group consisting of: ethyl, benzyl, phenyl, cyclohexylmethyl, 4-pyridinylmethyl, phenylethyl, 1H-indole-5-ylmethyl, 2-thienylmethyl, 2-furyl methyl, 4-fluorophenylmethyl, 4-chlorophenylmethyl, 4-bromophenylmethyl, 4-methoxyphenyl, 4-trifluoromethylphenylmethyl, 3,5-dimethoxyphenylmethyl, 4-carboxyphenylmethyl, cyclopentyl methyl, cyclobutylmethyl, 4-piperidylmethyl, 3-chlorophenylmethyl, 2-chlorophenylmethyl, and 4-tert butylphenylmethyl;
each R₂ is independently selected from the group consisting of: methyl;
n is 0, 1 or 2;
the method comprises the steps: 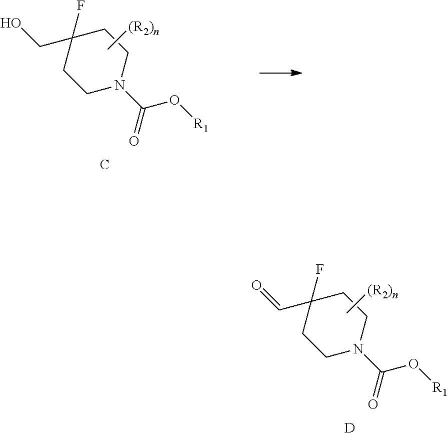
(1) In a solvent, converting compound C into compound D in the presence of an oxidizing agent;
wherein the oxidising agent is selected from the group consisting of: potassium chromate sulfate, pyridine chromium trioxide/pyridine, pyridine chromium trioxide, pyridine chlorochromic acid, oxalyl chloride/dimethylsulfoxide, pyridine sulfur trioxide/dimethylsulfoxide/triethylamine, Dess-Martin high iodine oxidiser, and iodosobenzene diacetate/TEMPO;
the solvent is selected from the group consisting of: C₁-C₄ halogenated hydrocarbon solvent, acetone, acetonitrile, tetrahydrofuran, ethyl acetate, toluene, dioxane, and combinations thereof.
